(19) United States
(12) Patent Application Publication
(10) Pub. No.: US 2013/0116333 A1
(43) Pub. Date: May 9, 2013

(54) SOLID TAPENTADOL IN NON-CRYSTALLINE FORM

(71) Applicants: Jana Paetz, Bonn (DE); Daniela Stumm, Berlin (DE); Wolfgang Albrecht, Ulm (DE); Alexandre Mathieu, Ulm (DE)

(72) Inventors: Jana Paetz, Bonn (DE); Daniela Stumm, Berlin (DE); Wolfgang Albrecht, Ulm (DE); Alexandre Mathieu, Ulm (DE)

(73) Assignee: RATIOPHARM GMBH, Ulm (DE)

(21) Appl. No.: 13/695,433

(22) PCT Filed: May 5, 2011
(86) PCT No.: PCT/EP2011/002247

§ 371 Date: Dec. 29, 2012

(30) Foreign Application Priority Data
May 5, 2010 (EP) 10004758.8

(51) Int. Cl. A61K 31/137

(52) U.S. Cl. CPC A61K 31/137
USPC 514/654; 564/374

(57) ABSTRACT

The invention relates to solid tapentadol in non-crystalline form together with a surface stabiliser in the form of a stable intermediate. In the intermediate of the invention, tapentadol is preferably present in amorphous form or in the form of a solid solution. The invention further relates to methods of producing tapentadol in a solid, non-crystalline form and to pharmaceutical formulations containing solid, non-crystalline tapentadol.

The invention relates to solid tapentadol in non-crystalline form together with a surface stabiliser in the form of a stable intermediate. In the intermediate of the invention, tapentadol is preferably present in amorphous form or in the form of a solid solution. The invention further relates to methods of producing tapentadol in a solid, non-crystalline form and to pharmaceutical formulations containing solid, non-crystalline tapentadol.

Tapentadol is an analgesic whose effect is based on two molecular mechanisms. First of all, like opioids, tapentadol activates μ-receptors and thus presynaptically and postsynaptically attenuates the transmission of pain stimuli in the spinal cord and brain. Secondly, tapentadol acts as a noradrenalin re-uptake inhibitor and thus increases the concentration of that nerve messenger in the synaptic gap.

In the context of this invention, the term “tapentadol” is understood to mean 3-(3-dimethylamino-1-ethyl-2-methyl-propyl)phenol in accordance with the following chemical formula (1).

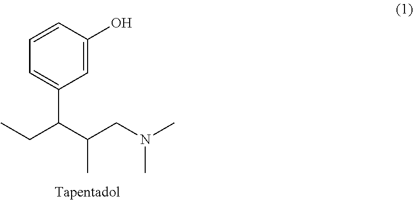 [SMILES: CCC(c1cccc(O)c1)C(C)CN(C)C]

3-(3-dimethylamino-1-ethyl-2-methyl-propyl)phenol has two centres of asymmetry, so that the compound can be present in the form of four different stereoisomers. In the context of this invention, 3-(3-dimethylamino-1-ethyl-2-methyl-propyl)phenol may be present as a mixture of all four diastereomers in any mixing ratio, but also as a mixture of two or three of the four stereoisomers or in stereoisomerically pure form. Preferred stereoisomers in this context are (+)-(1S,2S)-3-(3-dimethylamino-1-ethyl-2-methyl-propyl)phenol and (−)-(1R,2R)-3-(3-dimethylamino-1-ethyl-2-methyl-propyl) phenol, which can preferably be used as a 1:1 mixture (racemate) or particularly preferably in isomerically pure form. In particular, (1R,2R)-3-(3-dimethylamino-1-ethyl-2-methyl-propyl) phenol (hereinafter also referred to as “(1R,2R)-tapentadol)” according to formula (2) is used.

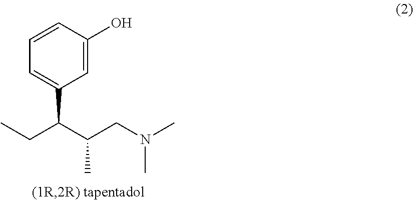 [SMILES: CC[C@@H](c1cccc(O)c1)[C@@H](C)CN(C)C]

In the context of this invention, tapentadol is usually employed in the form of the free base or in the form of a pharmaceutically acceptable salt. The salts may be acid addition salts. Examples of suitable salts are, inter alia, hydrochlorides (e.g. monohydrochloride). Tapentadol is preferably used in the form of the free base or in the form of the monohydrochloride. Tapentadol monohydrochloride is particularly preferred.

In particular, (1R,2R)-tapentadol monohydrochloride is used as the active agent in the context of this invention. Alternatively, (1R,2R)-tapentadol base is used as the active agent. Similarly, mixtures of the above-mentioned tapentadols can be used.

Synthesis pathways for crystalline tapentadol and its use as an analgesic have been described in EP 0 693 475 A1.

When developing pharmaceutical tapentadol formulations, however, the inventors of the present application were confronted with the fact that crystalline tapentadol can exist both as the free base and also as the hydrochloride in different crystalline, polymorphous forms. The individual polymorphs may not be stable, however, but tend to change into different crystalline, polymorphous forms. The frequently used tapentadol hydrochloride form A, for example, can change into form B under the influence of heat, see WO 2006/00441 A2. This process is reversible when the temperature is lowered. In addition, the polymorphous forms A, B and C of the tapentadol base described in WO 2009/071310 exhibit different solubility profiles.

In a patient, the different solubility profile leads to an undesirable, uneven rise in the concentration of the active agent. It was therefore an object of the present invention to provide stable tapentadol intermediates that can be processed into a dosage form which (even after storage) enables as even a rise as possible in the concentration in the patient. The aim was largely to avoid both inter-individual and also intra-individual deviations.

The objective of the present invention was therefore to overcome the above-mentioned disadvantages. The intention is to provide the active agent in a form possessing good flowability and thus making it possible to ensure good compression into tablets, even with solvent-free manufacturing processes. It is also the intention to provide the active agent in a form which does not have a tendency to agglomerate. In addition, it is intended to ensure an even distribution of the active agent. It is intended to avoid micronisation of the active agent.

The intention is also to provide dosage forms of tapentadol which ensure good solubility and bioavailability with good storage stability at the same time.

All the above-mentioned problems are supposed to be solved in particular for a high content of active agent (drug load), especially in order to ensure good patient compliance (when the patient obeys the doctor's treatment instructions). In addition, the problems are supposed to be solved both for a formulation designed for immediate release (or “IR” for short) and for modified release (or “MR” for short).

In the case of oral dosage forms for modified release (MR), the active agent should be released as completely as possible. For this reason, conventional delayed-release matrix formulations should be avoided, because they usually fail to release up to 20% of the active agent. In addition, in the case of the prior-art matrix tablets, a relatively large amount of excipients has to be used; that is not always advantageous and should be avoided. Furthermore, it is necessary in the case of prior-art MR formulations for high-viscosity polymers to be used. That is frequently not desirable, e.g. for toxicity reasons. The aim was therefore to provide a formulation for modified release avoiding large amounts of high-viscosity polymers.

It was unexpectedly possible to solve the problems by converting tapentadol, especially crystalline tapentadol, into a solid, non-crystalline form, especially a stabilised amorphous form, or into the form of a solid solution.

The subject matter of the invention is therefore tapentadol in solid, non-crystalline form, wherein the tapentadol is present together with a surface stabiliser. In the context of this application, two possible embodiments of tapentadol in solid, non-crystalline form are illustrated from this point of view.

In a first embodiment, the subject matter of the invention is therefore an intermediate containing amorphous tapentadol and a surface stabiliser. The intermediate is amorphous tapentadol in stabilised form.

In a second embodiment, the subject matter of the invention is an intermediate containing tapentadol in the form of a solid solution and a surface stabiliser. In this second embodiment, the surface stabiliser acts as a “matrix material”, in which tapentadol is present distributed in a molecularly disperse manner. The intermediate is a solid solution of tapentadol in stabilised form.

The subject matter of the invention is also various methods of producing solid, non-crystalline tapentadol in the form of the intermediate of the invention.

Finally, the subject matter of the invention comprises pharmaceutical formulations containing the solid, non-crystalline tapentadol of the invention or the stabilised tapentadol of the invention in the form of the intermediate of the invention.

The first embodiment of the present invention relates to amorphous tapentadol. The term “amorphous” is used in the context of this invention to designate the state of solid substances in which the components (atoms, ions or molecules, i.e. in the case of amorphous tapentadol the tapentadol molecules) do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order. In addition, an amorphous substance, especially amorphous tapentadol, usually has an average particle size of more than 300 nm.

In contrast to anisotropic crystals, solid amorphous substances are isotropic. Normally, they do not have a defined melting point, but instead gradually pass over into the liquid state after slowly softening. They can be distinguished from crystalline substances experimentally by means of X-ray diffraction, which does not reveal clearly defined interferences for them, but rather, in most cases, only a few diffuse interferences with small diffraction angles.

In the context of the first embodiment of this invention, the expression “amorphous tapentadol” preferably refers to a substance which consists of amorphous tapentadol. Alternatively, “amorphous tapentadol” may also contain small amounts of crystalline tapentadol components, provided that no defined melting point of crystalline tapentadol can be detected in DSC. A mixture containing 90 to 99.99% by weight amorphous tapentadol and 0.01 to 10% crystalline tapentadol is preferred, more preferably 95 to 99.9% by weight amorphous tapentadol and 0.1 to 5% crystalline tapentadol. The crystalline proportion is determined by means of quantitative X-ray diffractometry according to the method of Hermans and Weidinger.

In the context of this first embodiment of the invention, the tapentadol of the invention is present in stabilised form, namely in the form of an intermediate containing amorphous tapentadol and a surface stabiliser. In particular, the intermediate of the invention consists substantially of amorphous tapentadol and surface stabiliser. If—as described below—a crystallisation inhibitor is used in addition, the intermediate of the invention may consist substantially of amorphous tapentadol, surface stabiliser and crystallisation inhibitor. The expression “substantially” in this case indicates that small amounts of solvent etc. may also still be contained where applicable.

The second embodiment of the present invention relates to tapentadol in the form of a solid solution. The term “solid solution” is to be understood in the context of this invention as meaning that tapentadol is distributed in a molecularly disperse manner in a matrix which is present in a solid aggregate state at 25° C. and a pressure of 101 kPa.

It is preferable that in this second embodiment, the intermediate of the invention (containing tapentadol in the form of a solid solution) contains substantially no crystalline or amorphous tapentadol. In particular, the intermediate of the invention contains less than 15% by weight, more preferably less than 5% by weight, of amorphous or crystalline tapentadol, based on the total weight of the tapentadol present in the intermediate.

“Crystalline” generally means substances the smallest components of which build up crystal structures, but also substances consisting of tiny crystallites. The atoms, ions or molecules of which the respective crystal substance consists form characteristic arrangements which are repeated periodically, so that they exhibit a long-range order. Crystals are thus anisotropic. Crystalline substances can be identified experimentally by means of X-ray diffraction, which reveals clearly defined interference patterns for crystalline substances. In contrast to this, X-ray diffraction performed on amorphous substances does not reveal clearly defined interferences for them, but normally only a few diffuse interferences with small diffraction angles.

It is therefore preferable for “molecularly disperse” to be understood as meaning that X-ray diffraction analysis of the tapentadol contained in the embodiments of the invention does not reveal any clearly defined interference patterns, but at most only a few diffuse interferences with small diffraction angles.

It is further preferred that “molecularly disperse” should be understood as meaning that the intermediate of the invention does not contain any tapentadol particles with a particle size greater than 300 nm, more preferably greater than 200 nm, especially greater than 100 nm. The particle size is determined in this connection by means of confocal Raman spectroscopy. The measuring system preferably consists of an NTEGRA-Spektra Nanofinder ex NT-MDT.

In the context of this second embodiment of this invention, the solid solution of tapentadol of the invention is present in stabilised form, namely in the form of an intermediate containing molecularly disperse tapentadol and a surface stabiliser (as a matrix material). In particular, the intermediate of the invention consists substantially of molecularly disperse tapentadol and matrix material. If—as described below—a crystallisation inhibitor is used in addition, the intermediate of the invention may consist substantially of molecularly disperse tapentadol, surface stabiliser and crystallisation inhibitor. The expression “substantially” in this case indicates that small amounts of solvent etc. may also still be contained where applicable.

In a preferred embodiment of the intermediate of the invention, the surface stabiliser is used as a solid carrier for the non-crystalline tapentadol. The intermediate of the invention thus contains a surface stabiliser as a solid carrier, wherein non-crystalline tapentadol is applied to the solid carrier. Non-crystalline tapentadol is preferably applied to the surface stabiliser, i.e. non-crystalline tapentadol is adsorbed to the surface of the surface stabiliser, preferably substantially uniformly. The intermediate of the invention is thus preferably not a purely physical mixture of non-crystalline tapentadol and surface stabiliser.

Both embodiments of the present invention relate to an intermediate containing a surface stabiliser. The surface stabiliser is generally a substance which is suitable for stabilising tapentadol in amorphous form or in the form of a solid solution. The surface stabiliser is preferably a substance which is solid at 30° C. The surface stabiliser is preferably a polymer. In addition, the surface stabiliser also includes substances which behave like polymers. Examples of these are fats and waxes. Furthermore, the surface stabiliser also includes solid, non-polymeric compounds which preferably contain polar side groups. Examples of these are sugar alcohols or disaccharides.

A further subject matter of the invention is a method of identifying a pharmaceutical excipient which is suitable as a surface stabiliser for solid, non-crystalline (i.e. amorphous tapentadol or for tapentadol in the form of a solid solution) and which can hence be used for preparing the intermediate of the invention. The method comprises the steps of:

a) Providing a pharmaceutical excipient which is present in a solid aggregate state at 25° C. For this purpose, it is generally possible to choose the pharmaceutical excipients mentioned in the European Pharmacopoeia.
b) Twice in succession, heating up the solid excipient by means of DSC. In this case, two heating curves are recorded by means of DSC. The curves are usually recorded from 20° C. to no more than 20° C. below the decomposition range of the substance to be tested.

For this purpose a Mettler Toledo DSC 1 apparatus can be used. The work is performed at a heating rate of 1-20° C./min, preferably 5-15° C./min, and at a cooling rate of 5-25° C./min, preferably 10-20° C./min.

c) Selecting the excipient as “suitable” if a glass transition point of 20° C. to 120° C., preferably 25° C. to 100° C., can be seen in the second DSC heating curve.

Another subject matter of the invention is intermediates containing solid, non-crystalline tapentadol (i.e. amorphous tapentadol or tapentadol in the form of a solid solution) and a pharmaceutical excipient, selected by means of the method described above.

The surface stabiliser used for the preparation of the intermediate of the invention is preferably a polymer. The polymer to be used for the preparation of the intermediate preferably has a glass transition temperature (Tg) and/or a melting point of more than 20° C., preferably from 25° C. to 220° C., more preferably from 30° C. to 180° C., more preferably from 40° C. to 100° C. By means of immobilisation, a polymer with a Tg selected accordingly prevents the recrystallisation of the amorphous tapentadol or prevents the reversion of the molecular tapentadol dispersion into colloids or particles.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus can be used. The work is performed at a heating rate of 1-20° C./min, preferably 5-15° C./min, and at a cooling rate of 5-25° C./min, preferably 10-20° C./min.

In addition, the polymer which can be used for the preparation of the intermediate preferably has a weight-average molecular weight of 1,000 to 500,000 g/mol, more preferably from 2,000 to 120,000 g/mol, even more preferably 5,000 to 90,000 g/mol, especially 10,000 to 75,000 g/mol. If the polymer used for the preparation of the intermediate is dissolved in water in an amount of 2% by weight, the resulting solution usually has a viscosity of less than 3,000 mPa s, preferably from to 0.1 to 2,500 mPa s, more preferably from 0.5 200 mPa s, even more preferably from 1.5 to 20 mPa s, especially from 2.0 to 15 mPa s, measured at 20° C., and preferably determined in accordance with Ph. Eur., 6th edition, chapter 2.2.9 (capillary viscometer).

Hydrophilic polymers are preferably used to prepare the intermediate. This refers to polymers which possess hydrophilic groups. Examples of suitable hydrophilic groups are hydroxy, alkoxy, acrylate, methacrylate, sulphonate, carboxylate and quaternary ammonium groups. Hydroxy groups are preferable.

The intermediate of the invention may, for example, comprise the following hydrophilic polymers as the surface stabiliser: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC), e.g. L-HPC (low substituted hydroxypropyl cellulose); microcrystalline cellulose, polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF, or Povidon® VA64), polyalkylene glycols, such as polypropylene glycol or preferably polyethylene glycol, co-block polymers of polyethylene glycol, especially co-block polymers of polyethylene glycol and polypropylene glycol (Pluronic®, BASF) and mixtures of the polymers mentioned.

It is preferable that the polymers used as surface stabilisers should exhibit substantially no emulsifying effect. This means that the surface stabiliser used should preferably not contain any combination of hydrophilic and hydrophobic groups (especially hydrophobic fatty acid groups). In addition, it is preferable for the intermediate of the invention not to contain any polymers that have a weight-average molecular weight of more than 150,000 g/mol. It may happen that polymers of this kind have an undesirable influence on the dissolution characteristics.

Substances used particularly preferably as surface stabilisers are polyvinyl pyrrolidone, preferably with a weight-average molecular weight of 10,000 to 80,000 g/mol, especially 12,000 to 60,000 g/mol, a copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol, and HPMC, especially with a weight-average molecular weight of 20,000 to 90,000 g/mol and/or preferably a content of methyl groups of 10 to 35% and a content of hydroxy groups of 1 to 35%. In addition, microcrystalline cellulose can preferably be used, especially one with a specific surface area of 0.7-1.4 m/g. The specific surface area is determined by means of the gas adsorption method according to Brunauer, Emmet and Teller. The weight-average molecular weight is preferably determined by means of gel permeation chromatography. The copolymer of vinyl pyrrolidone and vinyl acetate preferably has the following structural unit.

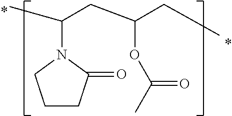

The copolymer of vinyl pyrrolidone and vinyl acetate illustrated particularly preferably has a weight-average molecular weight of 50,000 to 80,000 g/mol.

For the surface stabiliser, it is also particularly preferable to use co-block polymers of polyethylene glycol and polypropylene glycol, i.e. polyoxyethylene polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPa s, more preferably 500 to 1,500 mPa s, especially 800 to 1,200 mPa s.

In addition, the surface stabiliser also includes solid, non-polymeric compounds, which preferably contain polar side groups. Examples of these are sugar alcohols or disaccharides. Examples of suitable sugar alcohols and/or disaccharides are mannitol, sorbitol, xylitol, isomalt, glucose, fructose, maltose and mixtures thereof. The term “sugar alcohols” in this context also includes monosaccharides. In particular, mannitol, isomalt and sorbitol are used as the surface stabiliser.

Furthermore, the surface stabiliser may preferably also comprise silicates, more preferably magnesium aluminium silicates, even more preferably magnesium aluminium metasilicates, particularly preferably Al₂O₃.MgO.1,7SiO₂xH₂O (e.g. marketed as Neusilin®).

In a preferred embodiment, the intermediate of the invention contains solid, non-crystalline tapentadol (i.e. amorphous tapentadol or tapentadol in the form of a solid solution) and surface stabiliser, wherein the weight ratio of solid, non-crystalline tapentadol to surface stabiliser is 10:1 to 1:10, more preferably 5:1 to 1:3, even more preferably 3:1 to 1:2, especially 2:1 to 1:1.5. Tapentadol and surface stabiliser may, for example, be used in a ratio of 1:1.

In a particularly preferred embodiment, the intermediate of the invention contains solid, non-crystalline tapentadol hydrochloride (i.e. amorphous tapentadol hydrochloride or tapentadol hydrochloride in the form of a solid solution) and magnesium aluminium silicate as the surface stabiliser, preferably Al₂O₃.MgO.1,7SiO₂xH₂O, wherein the weight ratio of solid, non-crystalline tapentadol hydrochloride to the magnesium aluminium silicate is 5:1 to 1:3, more preferably 4:1 to 1:2, even more preferably 3:1 to 1:1.5, particularly preferably 2:1 to 1:1.4, especially 1.8:1 to 1:1.3. Tapentadol hydrochloride and magnesium aluminium silicate may, for example, be used in a ratio of 1:1, especially 1.0:1.0.

It is preferable that the type and quantity of surface stabiliser should be selected such that the resulting intermediate has a glass transition temperature (Tg) of more than 18° C., preferably more than 20° C., even more preferably more than 25° C. In addition, the resulting intermediate has a Tg of less than 180° C., more preferably less than 120° C., especially less than 80° C.

It is preferable that the type and quantity of the polymer should be selected such that the resulting intermediate is storage-stable. “Storage-stable” means that in the intermediate of the invention, after storage for 3 years at 25° C. and 50% relative humidity, the proportion of crystalline tapentadol-based on the total amount of tapentadol—is no more than 60% by weight, preferably no more than 30% by weight, more preferably no more than 15% by weight, in particular no more than 5% by weight.

It is advantageous for the surface stabiliser to be used in particulate form, wherein the volume-average particle size (D50) is less than 500 μm, preferably 5 to 250 μm, more preferably 25 to 150 μm.

In a preferred embodiment, in addition to solid, non-crystalline tapentadol (i.e. in addition to amorphous tapentadol or tapentadol in the form of a solid solution) and surface stabiliser, the intermediates of the invention also contain a crystallisation inhibitor based on an inorganic salt, an organic acid, a silicate or a polymer with a weight-average molecular weight (Mw) of more than 500,000 g/mol. These polymers which are suitable as crystallisation inhibitors are also referred to in the context of this invention as “high-viscosity polymers”. Their weight-average molecular weight is usually less than 5,000,000 g/mol. A preferred high-viscosity polymer is polyvinyl pyrrolidone (povidone).

The crystallisation inhibitor is preferably ammonium chloride, citric acid, magnesium aluminium metasilicate (especially marketed as Neusilin®) or Povidone K 90 (in accordance with Ph. Eur. 6.0).

The crystallisation inhibitor can generally be used in an amount of 1 to 30% by weight, preferably 2 to 25% by weight, more preferably 5 to 20% by weight, based on the total weight of the intermediate.

The intermediates of the invention are obtainable by a variety of preparation methods. Depending on the preparation method, the intermediates are obtained in different particle sizes. Normally, the intermediates of the invention are present in particulate form and have an average particle diameter (D50) of 1 to 750 μm, preferably 5 to 400 μm, depending on the respective preparation method.

The expression “average particle diameter” is determined in the context of this invention by means of laser diffractometry. In particular, a Malvern Instruments Mastersizer 2000 was used to determine the diameter (wet measurement with ultrasound for 60 sec., 2,000 rpm, the evaluation using the Fraunhofer method) and preferably using a dispersant in which the substance to be measured does not dissolve at 20° C.).

The “average particle diameter”, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

In a preferred embodiment, the intermediate of the invention, especially the intermediate containing non-crystalline tapentadol hydrochloride, has a water content of 0.01 to 15% by weight, more preferably 0.50% by weight to 12% by weight, even more preferably from 1.5 to 10% by weight, especially 4 to 9% by weight. The residual water content is determined according to the Karl Fischer method, using a coulometer at 160° C. A Metrohm 831 KF coulometer with a titration cell without a diaphragm is preferably used. Usually, a 20 mg sample of intermediate is analysed. It has unexpectedly been found that a deviation in the water content leads to an undesirably high recrystallisation rate.

It has been shown that a different water content would have a negative influence on the flowability and hence, in the case of a high content of active agent (drug load), also on the uniformity of the content (content uniformity).

Another subject matter of the invention is methods of preparing the intermediate of the invention. In the following, five preferred embodiments of such methods will be explained. Methods (1) to (3) here are preferable for the production of both amorphous tapentadol (=first embodiment of the intermediate of the invention) and also tapentadol in the form of a solid solution (second embodiment of the intermediate of the invention). Methods (4) and (5) are preferably used to produce amorphous tapentadol.

In particular, method (3) is used to produce amorphous tapentadol and/or tapentadol in the form of a solid solution.

In a first preferred method, the invention relates to a “pellet-layering process”, i.e. a method of preparing an intermediate of the invention, comprising the steps of

(a1) dissolving the tapentadol and the surface stabiliser in a solvent or mixture of solvents, and
(b1) spraying the solution from step (a1) onto a substrate core.

In step (a1), tapentadol and the surface stabiliser described above are dissolved, preferably completely dissolved, in a solvent or mixture of solvents. It is preferable to use crystalline tapentadol for this purpose. In addition, it is preferable for tapentadol to be used in the form of one of the acid addition salts described above; tapentadol monohydrochloride, for example, can advantageously be used. Alternatively, tapentadol base may also be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers in this first method are in particular modified celluloses, such as HPMC (preferably with a weight-average molecular weight of 20,000 to 90,000 g/mol), sugar alcohols, such as mannitol isomalt and sorbitol, and polyethylene glycol, in particular polyethylene glycol with a molecular weight of 2,000 to 10,000 g/mol. Also, a copolymer of vinyl pyrrolidone and vinyl acetate, especially with a weight-average molecular weight of 50,000 to 80,000 g/mol is preferably used.

If the intermediate to be prepared is additionally intended to contain a crystallisation inhibitor based on an inorganic salt or an organic acid, or a highly viscous polymer, this can likewise be added in step (a1). Reference is made to the above observations with regard to the type and amount of the crystallisation inhibitor.

In step (b1), the solution from step (a1) is sprayed onto a substrate core. Suitable substrate cores are particles consisting of pharmaceutically acceptable excipients, especially “neutral pellets”. The pellets preferably used are those which are available under the trade name Cellets® and which contain a mixture of lactose and microcrystalline cellulose, or sugar spheres, which are a mixture of starch and sugar.

Step (b1) is preferably performed in a fluidised bed dryer, such as a Glatt GPCG 3 (Glatt GmbH, Germany). Work is preferably performed with air inlet temperatures of 50 to 100° C., preferably von 60 to 80° C., with product temperatures of 25 to 50° C., preferably 30 to 40° C. and with a spray pressure of 0.9 to 2.5 bar, preferably 1 to 1.5 bar.

Depending on the choice of starting materials in step (a1) and the process parameters in step (b1), the resulting intermediate may contain tapentadol in amorphous form or in the form of a solid solution.

The process conditions in this first method are preferably selected such that the resulting intermediate particles have a volume-average particle diameter (D50) of 50 to 800 μm, more preferably 150 to 550 μm, especially 180 to 350 μm.

In a second preferred embodiment, the invention relates to a spray-drying method of preparing the intermediate of the invention, comprising the steps of
(a2) dissolving tapentadol and the surface stabiliser in a solvent or mixture of solvents, and
(b2) spray-drying the solution from step (a2).

In step (a2), tapentadol and the matrix material described above are dissolved, preferably completely dissolved, in a solvent or mixture of solvents. It is preferable to use crystalline tapentadol. In addition, it is preferable for tapentadol to be used in the form of one of the acid addition salts described above; tapentadol dihydrochloride, for example, can advantageously be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, an ethanol/water mixture is used.

Suitable surface stabilisers in this method are in particular modified celluloses, such as HPMC (preferably with a weight-average molecular weight of 20,000 to 90,000 g/mol), polyvinyl pyrrolidone and copolymers thereof, e.g. copolymers with vinyl acetate, wherein polyvinyl pyrrolidone or copolymers thereof preferably have a weight-average molecular weight of 20,000 to 80,000 g/mol, and sugar alcohols, such as mannitol, isomalt and sorbitol.

If the intermediate to be prepared is additionally intended to contain a crystallisation inhibitor based on an inorganic salt or an organic acid, or a highly viscous polymer, this can likewise be added in step (a2). Reference is made to the above observations with regard to the type and amount of the crystallisation inhibitor.

In the subsequent step (b2), the solution from step (a2) is spray-dried. The spray-drying is usually carried out in a spray tower. As an example, a Búchi B-191 is suitable (Büchi Labortechnik GmbH, Germany). Preferably an inlet temperature of 100° C. to 150° C. is chosen. The amount of air is, for example, 500 to 700 litres/hour, and the aspirator preferably runs at 80 to 100%.

Depending on the choice of starting materials in step (a2) and the process parameters in step (b2), the resulting intermediate may contain tapentadol in amorphous form or in the form of a solid solution.

The process conditions in this second method are preferably selected such that the resulting intermediate particles have a volume-average particle diameter (D50) of 1 to 250 μm, more preferably 2 to 100 μm, even more preferably 3 to 50 μm, especially 4 to 25 μm. In a further preferred embodiment, one or more excipients, especially fillers such as microcrystalline cellulose, may be added during the spray-drying. In this case, the resulting intermediate particles have a volume-average particle diameter (D50) of 1 to 250 μm, more preferably 2 to 150 μm, even more preferably 5 to 120 μm, especially 10 to 90 μm.

In a third preferred embodiment, the invention relates to a melt-processing method, preferably a melt-extrusion process, i.e. a method of preparing the intermediate of the invention, comprising the steps of
(a3) mixing tapentadol and surface stabiliser, and
(b3) melt-processing, preferably melt-extruding, the mixture, where the melt-processing, preferably extrusion, conditions are selected such that there is a transition from crystalline to non-crystalline tapentadol.

In step (a3), crystalline tapentadol is mixed with the surface stabiliser, preferably in a mixer. In this version of the method of the invention, a matrix material (i.e. a surface stabiliser) in polymeric form is preferably used. In addition, tapentadol is preferably used in the form of the free base. Alternatively, tapentadol HCl, for example, may also be used.

Suitable polymeric surface stabilisers in this third method are especially polyvinyl pyrrolidone and vinyl pyrrolidone/vinyl acetate copolymers, and also polyvinyl alcohols, methacrylates, PEG and HPMC. The weight-average molecular weight of the polymers used is usually 4,000 to 80,000 g/mol, preferably 6,000 to 80,000 g/mol.

If the intermediate to be prepared is additionally intended to contain a crystallisation inhibitor based on an inorganic salt or an organic acid, or a highly viscous polymer, this can likewise be added in step (a3). Reference is made to the above observations with regard to the type and amount of the crystallisation inhibitor.

In step (b3), the mixture is melt-processed, preferably extruded. In the course of the melt-processing (b3), tapentadol is processed with the—preferably polymeric, especially thermoplastic—surface stabiliser in such a way that tapentadol is embedded in the surface stabiliser in non-crystalline form. The melt processing can preferably be carried out as melt granulation or melt extrusion.

The mixture from step (a3) is conventionally processed in the extruder into a homogeneous melt. The extrusion conditions are preferably selected such that there is a transition from crystalline to amorphous tapentadol.

The extruders used may be conventional melt extruders, such as a Leistritz Micro 18. The melt-processing temperature or extrusion temperature depends on the nature of the matrix material. It usually lies between 80 and 250° C., preferably between 100 and 180° C., especially between 105 and 150° C. The extrusion is preferably carried out at an outlet pressure of 10 bar to 100 bar, more preferably at 20 to 80 bar.

The cooled melt is usually comminuted by a rasp screen (e.g. Comil® U5) and in this way accordingly reduced to a uniform particle size.

It has unexpectedly been found that the particle size of the resulting intermediate (especially in the case of melt extrusion, but also intermediates obtained by the other methods of the invention) has a considerable influence on the release characteristics.

Hence, it is preferable for intermediates used for a modified-release pharmaceutical formulation to be screened with a screen with a mesh width of more than 0.71 mm. In particular, screens are used here with a mesh width of more than 0.71 mm to 1.5 mm.

Similarly, it is preferable for intermediates used for an immediate-release pharmaceutical formulation to be screened with a screen with a mesh width of 0.71 mm or less. In particular, screens are used here with a mesh width of 0.4 to 0.71 mm.

Depending on the choice of starting materials in step (a3) and the process parameters in step (b3), the resulting intermediate may contain tapentadol in amorphous form or in the form of a solid solution. In particular, it has proven suitable for the extruder to be equipped with a kneader unit if tapentadol is to be obtained in the form of a solid solution. The kneader unit should be designed such that intensive blending is ensured, so that a solution of tapentadol in the surface stabiliser is ensured.

The process conditions in this third method are preferably selected such that the resulting intermediate particles have a volume-average particle diameter (D50) of 150 to 1,000 μm, more preferably a D50 of 200 to 600 μm.

Instead of granulating the extruded material, “direct injection moulding” may also be performed. In this case, the method of the invention includes the step of
(c3) injection moulding the extruded material into moulds for pharmaceutical dosage forms.

### Examples are Moulds for Tablets.

In a fourth preferred embodiment, the invention relates to a freeze-drying process, i.e. a method of preparing the intermediate of the invention, comprising the steps of
(a4) dissolving the tapentadol, preferably the crystalline tapentadol and the surface stabiliser, in a solvent or mixture of solvents, and
(b4) freeze-drying the solution from step (a4).

In step (a4), tapentadol, preferably crystalline tapentadol and the surface stabiliser described above, is dissolved, preferably completely dissolved, in a solvent or mixture of solvents. In addition, it is preferable for tapentadol to be used in the form of one of the acid addition salts described above; tapentadol monohydrochloride, for example, can advantageously be used. Alternatively, tapentadol base may also be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers in this method are especially modified celluloses such as HPMC (preferably with a weight-average molecular weight of 20,000 to 90,000 g/mol) and sugar alcohols such as isomalt, mannitol and sorbitol.

If the intermediate to be prepared is additionally intended to contain a crystallisation inhibitor based on an inorganic salt or an organic acid, or a highly viscous polymer, this can likewise be added in step (a4). Reference is made to the above observations with regard to the type and amount of the crystallisation inhibitor.

The solution from step (a4) is cooled to about 10 to 50° C. below freezing point (i.e. it is frozen). Then the solvent is removed by sublimation. This is preferably done when the conductivity of the solution is less than 2%. The sublimation temperature is preferably determined by the point of intersection of the product temperature and Rx −10° C. Sublimation is preferably effected at a pressure of less than 0.1 mbar.

After completion of the sublimation, the lyophilised intermediate is heated to room temperature.

The process conditions in this fourth method are preferably selected such that the resulting intermediate particles have a volume-average particle diameter (D50) of 0.5 to 250 μm, more preferably 1 to 150 μm, especially 5 to 100 μm.

In a fifth preferred embodiment, the invention relates to a milling process, i.e. a method of preparing the intermediate of the invention, comprising the steps of
(a5) mixing tapentadol, preferably crystalline tapentadol, and surface stabiliser, and
(b5) milling the mixture from step (a5), the milling conditions preferably being selected such that there is a transition from crystalline to non-crystalline, preferably amorphous tapentadol.

Crystalline tapentadol and surface stabiliser are preferably mixed in step (a5). The mixture is milled in step (b5). The mixing may take place before or even during the milling, i.e. steps (a5) and (b5) may be performed simultaneously.

If the intermediate to be prepared is additionally intended to contain a crystallisation inhibitor based on an inorganic salt or an organic acid, this can likewise be added in step (a5) or (b5). Reference is made to the above observations with regard to the type and amount of the crystallisation inhibitor.

The milling conditions are preferably selected such that there is a transition from crystalline to amorphous tapentadol.

The milling is generally performed in conventional milling apparatuses, preferably in a ball mill, such as a Retsch® PM 100.

The milling time is usually 10 minutes to 10 hours, preferably 30 minutes to 8 hours, more preferably 2 hours to 6 hours.

Suitable surface stabilisers in this fifth method are in particular polyvinyl pyrrolidone, modified celluloses, such as HPMC, sugar alcohols, such as isomalt and sorbitol, and polyethylene glycol, especially polyethylene glycol with a molecular weight of 2,000 to 10,000 g/mol.

The process conditions in this fifth method are preferably selected such that the resuiting intermediate particles have a volume-average particle diameter (D50) of 0.1 to 350 μm, more preferably 1 to 120 μm, especially 5 to 90 μm.

In a sixth preferred embodiment, the invention relates to a method of preparing the intermediate of the invention, comprising the steps of
(a6) dissolving the tapentadol, preferably the crystalline tapentadol, in a solvent or mixture of solvents,
(b6) adding the surface stabiliser and
(c6) removing the solvent or mixture of solvents, wherein tapentadol is preferably adsorbed to the surface of the surface stabiliser in non-crystalline form.

In step (a6), tapentadol, preferably crystalline tapentadol, is dissolved, preferably completely dissolved, in a solvent or mixture of solvents. The dissolution of the tapentadol is preferably achieved by stirring, such as with the stirring devices known from the state of the art.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and alcohol, preferably ethanol and/or isopropanol, is used.

In step (b6), the surface stabiliser is added, preferably by stirring with the above-mentioned stirring devices. The surface stabiliser is preferably added in solid form. In the process, a solution or preferably a suspension can form. The surface stabiliser can preferably also be added in batches.

Step (b6) may comprise the further stirring of the solution or suspension formed. This is preferably done in order to obtain a homogeneous distribution of the components.

Suitable surface stabilisers in this method are in particular magnesium aluminium silicates such as Al₂O₃.MgO.1,7SiO₂xH₂O, sugar alcohols, such as mannitol, isomalt and sorbitol, and polyethylene glycol, especially polyethylene glycol with a molecular weight of 2,000 to 10,000 g/mol. Also, a copolymer of vinyl pyrrolidone and vinyl acetate, especially with a weight-average molecular weight of 50,000 to 80,000 g/mol, or polyvinyl pyrrolidone, preferably with a weight-average molecular of von 10,000 to 80,000 g/mol is preferably used. Magnesium aluminium silicates are particularly preferable. The use of magnesium aluminium silicate in this embodiment leads to an intermediate with particularly good flowability.

The solvent or mixture of solvents (c6) can be removed by heating, preferably heating to the boiling point of the solvent or mixture of solvents or just above that temperature, so that they evaporate. Alternatively, the solvent or mixture of solvents can be evaporated at a reduced pressure. Additionally, the solvent or mixture of solvents can be evaporated by heating and at reduced pressure. In order to evaporate the solvent or mixture of solvents, it is possible to use the equipment known in the state of the art, such as the Rotavapor® R-210/R215 ex Büchi or the Laborota 20 large rotation evaporator ex Heidolph.

Steps (a6), (b6) and (c6) are preferably carried out such that tapentadol is “deposited” on the surface stabiliser. This means that steps (a6), (b6) and (c6) are preferably carried out such that non-crystalline tapentadol is adsorbed, preferably substantially uniformly adsorbed, to the surface of the surface stabiliser.

The intermediate of the invention (i.e. the stabilised non-crystalline tapentadol of the invention) is usually employed to prepare a pharmaceutical formulation.

An “intermediate” in the context of the present invention is understood to mean a pharmaceutical composition which is not present in the form of a dosage form to be administered.

In order to prepare a dosage form to be administered (also referred to in this application as a “pharmaceutical formulation”) the intermediate may, for example, be filled in sachets or capsules or preferably compressed into tablets. The processing of the intermediate into a pharmaceutical formulation can be carried out with or without the addition of pharmaceutical excipients. Excipients are preferably added.

The subject matter of the invention is therefore a pharmaceutical formulation containing intermediate of the invention and pharmaceutical excipients.

These are the excipients with which the person skilled in the art is familiar, such as those which are described in the European Pharmacopoeia.

Examples of excipients used are disintegrants, anti-stick agents, emulsifiers, pseudoemulsifiers, fillers, additives to improve the powder flowability, glidants, wetting agents, gel-forming agents and/or lubricants. Where appropriate, further excipients can also be used.

The ratio of active agent to excipients is preferably selected such that the resulting formulations contain

1 to 80% by weight, more preferably 5 to 60% by weight, especially 10 to 40% by weight non-crystalline tapentadol, and
20 to 99% by weight, more preferably 40 to 95% by weight, in particular 60 to 90% by weight pharmaceutically acceptable excipients.

In these ratios specified, the amount of surface stabiliser used to prepare the intermediate of the invention is counted as an excipient. This means that the amount of active agent refers to the amount of non-crystalline tapentadol contained in the formulation.

It has been shown that the intermediates of the invention are suitable for serving both as a basis for a dosage form with immediate release (or “IR” for short) and also for modified release (or “MR” for short).

In a preferred embodiment for an IR formulation, a relatively large amount of disintegrant is used. In that preferred embodiment, the pharmaceutical formulation of the invention therefore contains

1 to 30% by weight, more preferably 3 to 15% by weight, especially 5 to 12% by weight disintegrant, based on the total weight of the formulation.

In addition, it is preferable for this IR formulation to contain intermediate of the invention which has been screened with a screen with a mesh width of 0.71 mm or less. For this IR formulation, the intermediate was preferably likewise produced by means of melt granulation.

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone. Alkaline disintegrants can likewise be used. The term “alkaline disintegrants” means disintegrants which, when dissolved in water, produce a pH level of more than 7.0.

More preferably, inorganic alkaline disintegrants are used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Crospovidone or sodium hydrogen carbonate is particularly preferably used as a disintegrant, especially in the above-mentioned amounts.

In a preferred embodiment for an MR formulation, a relatively small amount of disintegrant is used. In that preferred embodiment, the pharmaceutical formulation of the invention therefore contains

0 to 10% by weight, more preferably 0.5 to 8% by weight, especially 1 to 5% by weight disintegrant, based on the total weight of the formulation.

In addition, it is preferable for this MR formulation to contain intermediate of the invention which has been screened with a screen with a mesh width of more than 0.71 mm. For this MR formulation, the intermediate was preferably likewise produced by means of melt granulation.

In the case of the MR formulation, croscarmellose or crospovidone is preferred as the disintegrant.

Furthermore, the pharmaceutical formulation (both for IR and for MR) preferably contains one or more of the excipients mentioned in the European Pharmacopoeia. These will be explained in more detail below.

The formulation of the invention preferably contains fillers. “Fillers” generally means substances which serve to form a quantity that is good to process, especially to form the body of the tablet in the case of tablets with small amounts of active agent (e.g. less than 70% by weight). This means that fillers “dilute” the active agents in order to produce an adequate compound, especially a tablet-compression mixture.

Examples of preferred fillers are starch, starch derivatives, treated starch, talcum, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Similarly, siliconated microcrystalline cellulose (Prosolv® Rettenmaier & Sóhne, Germany) can be used.

Fillers are normally used in an amount of 0 to 40% by weight, more preferably 1 to 25% by weight, based on the total weight of the formulation.

In addition, excipients can be used to improve the powder flowability. One example of an additive to improve the powder flowability is dispers silicon dioxide, e.g. known under the trade name Aerosil®. Preferably, silica is used with a specific surface area of 50 to 400 m²/g, especially 100 to 250 m²/g, determined by gas adsorption in accordance with Ph. Eur., 6th edition 2.9.26.

Additives to improve the powder flowability are generally used in an amount of 0.1 to 3% by weight, based on the total weight of the formulation.

Lubricants can be used in addition. Lubricants are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate (Pruv®) and/or magnesium stearate.

Lubricants are generally used in an amount of 0.1 to 5% by weight, preferably 0.5 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. Mannitol, for example—if used as a surface stabiliser—is not also counted as a filler in addition.

The pharmaceutical formulation of the invention is preferably pressed into tablets. For the products currently marketed under the name Nucynta®, wet granulation is used for this purpose.

It has, however, become apparent that the properties of the resulting tablets (e.g. with regard to the stability of the active agent) can be improved if wet granulation is avoided.

The intermediates of the invention are therefore compressed into tablets by means of direct compression or are subjected to dry granulation before being compressed into tablets. Intermediates with a bulk density of less than 0.5 g/ml are preferably processed by dry granulation.

Direct compression is especially preferred if the intermediate is prepared by means of melt extrusion (process steps (a3) and (b3) or pellet layering (process steps (a1) and (b1)).

Dry granulation is particularly preferable if the intermediate is prepared by means of spray drying (process steps (a2) and (b2)), freeze drying (process steps (a4) and (b4)) or milling (process steps (a5) and (b5)).

A further aspect of the present invention therefore relates to a dry granulation process comprising the steps of

(I) preparing the intermediate of the invention and one or more pharmaceutical excipients (especially those described above);
(II) compacting it into a slug; and
(III) granulating or comminuting the slug.

In step (I), the intermediate of the invention and excipients are preferably mixed. The mixing can be performed in conventional mixers. Alternatively, it is possible that the tapentadol intermediate is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the method of the invention, the mixture from step (I) is compacted into a slug. It is preferable here that it should be dry compacting, i.e. the compacting is preferably performed in the absence of solvents, especially in the absence of organic solvents.

The compacting conditions are usually selected such that the intermediate of the invention is present in the form of a slug of compacted material, the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 5 to 70 kN/cm roll width, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

In step (III) of the method, the slug is granulated. The granulating can be performed with processes known in the state of the art.

In a preferred embodiment, the granulation conditions are selected such that the resulting particles (granules) have a volume-average particle size ((D₅₀) value) of 50 to 800 μm, more preferably 100 to 650 μm, even more preferably 130 to 500 μm, especially 180 to 350 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

The granules resulting from step (III) can be further processed into pharmaceutical dosage forms. For this purpose, the granules are filled into sachets or capsules, for example. The granules resulting from step (III) are preferably pressed into tablets (=step IV).

In step (IV) of the method, the granules obtained in step (III) are pressed into tablets, i.e. the step involves compression into tablets. The compression can be performed with tableting machines known in the state of the art. Eccentric presses or rotary presses are preferably used. In the case of rotary presses, a compressive force of 2 to 40 kN, preferably 2.5 to 35 kN, is usually applied. In the case of eccentric presses, a compressive force of 1 to 20 kN, preferably 2.5 to 10 kN, is usually applied. By way of example, the Riva Piccola is used.

In step (IV) of the method, pharmaceutical excipients may optionally be added to the granules from step (III).

The amounts of excipients added in step (IV) usually depend on the type of tablet to be produced and the amount of excipients which were already added in steps (I) or (II).

In the case of direct compression, only steps (I) and (IV) of the method described above are performed.

The tableting conditions are preferably selected such that the resulting tablets have a ratio of tablet height to weight of 0.005 to 0.3 mm/mg, particularly preferably 0.01 to 0.2 mm/mg.

The method of the invention is preferably performed such that the tablet of the invention contains tapentadol in an amount of more than 20 mg to 50 mg, more preferably from 30 mg to 350 mg, especially 50 mg to 250 mg. Hence, the subject matter the invention comprises tablets containing 50 mg, 75 mg, 100 mg, 125 mg, 150 mg, 175 mg, 200 mg, 225 mg, 250 mg, 275 mg, 300 mg, 325 mg or 350 mg tapentadol in non-crystalline form.

The resulting tablets preferably have a hardness of 50 to 300 N, particularly preferably 80 to 250 N, especially 100 to 220 N. The hardness is determined in accordance with Ph. Eur. 6.0, section 2.9.8.

In addition, the resulting tablets preferably have a friability of less than 3%, particularly preferably less than 2%, especially less than 1%. The friability is determined in accordance with Ph. Eur. 6.0, section 2.9.7.

Finally, the tablets of the invention usually have a “content uniformity” of 95 to 105% of the average content, preferably 98 to 102%, especially 99 to 101%. (This means that all the tablets have a content of active agent of between 95 and 105%, preferably between 98 and 102%, especially between 99 and 101% of the average content.) The “content uniformity” is determined in accordance with Ph. Eur. 6.0, section 2.9.6.

In the case of an IR formulation, the release profile of the tablets of the invention according to the USP method (type II, paddle, 0.1nHCl, 37° C., 75 rpm) after 10 minutes usually indicates a content released of at least 30%, preferably at least 60%, especially at least 90%.

In the case of an MR formulation, the release profile of the tablets of the invention according to the USP method (type II, paddle, 0.1nHCl, 37° C., 75 rpm) after 60 minutes usually indicates a content released of 10%, preferably 20%, especially 30%.

The above details regarding hardness, friability, content uniformity and release profile preferably relate here to the non-film-coated tablet for an IR formulation. For a modified-release tablet, the release profile relates to the total formulation.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art may be employed. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack or natural gum, such as carrageenan.

The thickness of the coating is preferably 1 to 100 μm, especially 5 to 75 μm.

The pharmaceutical formulations of the invention are usually characterised by a release and absorption that lead to advantageous figures for the AUC (“area under curve”), the area under the curve of the plasma level 0 to 48 hours after peroral administration), advantageous figures for the Cₘₐₓ (maximum plasma level) and advantageous figures for the Tₘₐₓ (time when the maximum plasma level is reached after peroral administration).

In a preferred embodiment, the peroral administration of the formulations of the invention to a human patient leads to a plasma level profile characterised by a Tₘₐₓ regarding the active agent tapentadol of about 0.5 to 7.0, preferably 1.0 to 6.0 hours for MR formulations.

In a preferred embodiment, the peroral administration of the formulations of the invention to a human patient leads to a plasma level profile characterised by a Cₘₐₓ regarding the active agent tapentadol of about 35 to 210 ng/ml, preferably 40 to 180 ng/ml for IR formulations and 5 to 90 ng/ml, preferably 10 to 60 ng/ml for MR formulations.

In a preferred embodiment, the peroral administration of the formulations of the invention to a human patient leads to a plasma level profile characterised by an AUC regarding the active agent tapentadol of about 100 to 1,000 ng·h/ml, preferably 130 to 850 ng·h/ml for IR formulations and about 40 to 850 ng·h/ml, preferably 50 to 800 ng·h/ml for MR formulations.

The above-mentioned plasma level figures are preferably averages, obtainable by examining blood samples from a group of 10 candidates (with an average body weight of 70 kg), the corresponding blood samples being taken 0, 1, 3, 4, 6, 8, 24 and 48 hours after the peroral administration of the formulation of the invention. The figures are preferably determined as described in Bauer, Frömming, Führer “Lehrbuch der pharmazeutischen Technologie” (Textbook of pharmaceutical technology), 8th edition, 2006, chapter 7.4, especially pages 207 to 214.

In a preferred embodiment, the pharmaceutical formulations of the invention are used as an analgesic, e.g. for the treatment of chronic back pain. It is particularly preferable to treat groups of patients suffering from blood pressure or heart rhythm disorders.

The subject matter of the invention is thus also a tablet containing 50 to 500 mg 30 tapentadol, the tablet having a hardness of 50 to 250 N, a friability of less than 3% and a content uniformity of 95 to 105%, and wherein the administration regarding the active agent tapentadol leads to a Tₘₐₓ of 0.5 to 6 hours, preferably 1 to 5 hours, a C. of 5 to 210 ng/ml, preferably 10 to 180 ng/ml, and an AUC of 40 to 1,000 ng·h/ml, preferably 50 to 800 ng·h/ml. In the tablet of the invention, tapentadol is preferably present in the form of the intermediate of the invention. The tablet of the invention is preferably administered once or twice daily.

The invention will now be illustrated with reference to the following examples.

### EXAMPLES

### Example 1a

### Preparation of an Intermediate Containing Amorphous Tapentadol HCl

0.2 g crystalline (1R,2R)-tapentadol hydrochloride (form A) were dissolved by stirring in 2 ml water and 12 ml isopropanol. 0.2 g Al₂O₃.MgO.1,7SiO₂xH₂O (Neusilin®) were added to this, and the suspension was stirred for 5 minutes at 23° C. After the mixture of solvents was removed in a rotation evaporator, the intermediate was present as a white solid.

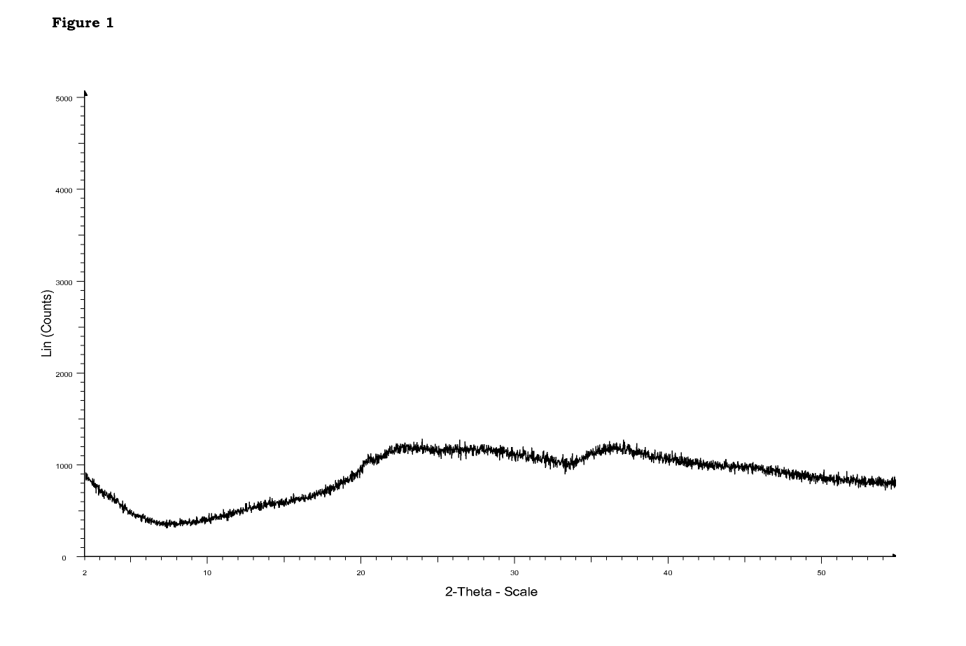

In XRPD, no clearly defined interference patterns, but only a few diffuse interferences could be detected, see FIG. 1. The conclusion drawn from this was that the tapentadol HCl was present in an amorphous structure.

The measurements for the X-ray diffractogram were carried out on a D8 ADVANCE X-ray diffractometer for powder diffractometry applications ex Bruker-AXS, Karlsruhe, Germany, and analysed with Bruker-AXS's EVA program. The following measuring conditions were observed:

radiation: CuKα
source: 38 KV/40 mA
2Θ−range/°: 2≦2Θ≦55
step size/°: 0.017

### Example 1b

### Preparation of an Intermediate Containing Amorphous Tapentadol HCl

2 g crystalline (1R,2R)-tapentadol hydrochloride (form A) were dissolved by stirring in 10 ml water and 10 ml isopropanol. 0.2 g Al₂O₃.MgO.1,7SiO₂xH₂O (Neusilin®) were added to this, and the suspension was stirred for 10 minutes at 23° C. After the mixture of solvents was removed in a rotation evaporator, the intermediate was present as a white solid.

In XRPD, no clearly defined interference patterns, but only a few diffuse interferences could be detected. The conclusion drawn from this was that the tapentadol HCl was present in an amorphous structure.

### Example 1c

### Preparation of an Intermediate Containing Amorphous Tapentadol HCl

0.2 g crystalline (1R,2R)-tapentadol hydrochloride (form A) were dissolved by stirring in 3 ml water and 10 ml isopropanol. 0.2 g polyvinyl pyrrolidone (Kollidon® 30) were added to this, and the suspension was stirred for 5 minutes at 23° C. After the mixture of solvents was removed in a rotation evaporator, the intermediate was present as a white solid.

In XRPD, no clearly defined interference patterns, but only a few diffuse interferences could be detected. The conclusion drawn from this was that the tapentadol HCl was present in an amorphous structure.

### Example 2a

### Preparation of the Intermediate Containing Amorphous Tapentadol HCl by Lyophilisation

The following batch for 100 dosage forms was produced.

5 g crystalline (1R,2R)-tapentadol hydrochloride were dissolved in water/ethanol together with 3 g mannitol. That solution was reduced in temperature to −55° C. and frozen. Once the conductivity had reached less than 2%, the frozen mixture, at a temperature determined by the point of intersection between the product temperature and Rx −10° and at a pressure of less than 0.1 mbar, was dried, or the solvent was removed by sublimation.

After drying, the lyophilised material was heated to room temperature (20-25° C.).

It was possible to carry out the further processing in accordance with Examples 5 or 6.

### Example 2b

### Preparation of the Intermediate Containing Amorphous Tapentadol Base by Lyophilisation

The following batch for 20 dosage forms was produced.

1 g crystalline (1R,2R)-tapentadol base was dissolved in water/ethanol together with 5 g HPMC. That solution was reduced in temperature to −55° C. and frozen. Once the conductivity had reached less than 2%, the frozen mixture, at a temperature determined by the point of intersection between the product temperature and Rx −10° and at a pressure of less than 0.1 mbar, was dried, or the solvent was removed by sublimation.

After drying, the lyophilised material was heated to room temperature (20-25° C.).

It was possible to carry out the further processing in accordance with Examples 5 or 6.

### Example 3a

### Preparation of the Intermediate Containing Tapentadol Base in the Form of a Solid Solution by Melt Extrusion, Especially for IR Formulations

The following batch for 10,000 dosage forms was produced.

500 g (1R,2R)-tapentadol base (in crystalline form) were extruded in a Leistritz micro 18 melt extruder together with 800 g Povidon® VA64 and with a temperature cascade of 90-180° C. The twin-screw extruder was equipped with various screw elements. A kneading unit was installed in order to ensure the necessary thorough mixing and dissolution of the tapentadol in the polymer (surface stabiliser). The strands of extruded material were cooled.

Further processing was performed after screening on a Comil® U5 (0.50 mm) in accordance with Example 6a.

### Example 3b

### Preparation of the Intermediate Containing Tapentadol Base in the Form of a Solid Solution by Melt Extrusion, Especially for MR Formulations

The following batch for 10,000 dosage forms was produced.

500 g (1R,2R)-tapentadol base (in crystalline form) were extruded in a Leistritz micro 18 melt extruder together with 800 g Povidon® VA64 and with a temperature cascade of 90-180° C. The twin-screw extruder was equipped with various screw elements. A kneading unit was installed in order to ensure the necessary thorough mixing and dissolution of the tapentadol in the polymer (surface stabiliser). The strands of extruded material were cooled.

Further processing was performed after screening on a Comil® U5 (1.00 mm) in accordance with Example 6b.

### Example 4

### Preparation of the Intermediate by Spray-Drying

The following batch for 100 dosage forms was produced.

5 g crystalline (1R,2R)-tapentadol base were dissolved in water/ethanol together with 4 g HPMC and 0.5 g citric acid and spray-dried on a Büchi TYP B 191 spray tower The following parameters were maintained in the process:

temperature 130° C., spray rate 5-20%, aspirator power 35-90%, flow control 300-700 l/h

The spray-dried material underwent a final drying stage for 24 h at 30° C. in a tray drying cabinet.

By adding microcrystalline cellulose to the spray suspension, it was possible to influence the release properties positively.

Further processing was performed after screening on a Comil® U5 (0.71 mm) in accordance with Example 5.

### Example 5

### Production of Tablets by Means of Dry Granulation

In order to produce tablets, the following formulation was used.

| | |
|---|---|
| 1. Intermediate according to Example 4 | 95 mg |
| 2. Prosolv ® 90 | 230 mg |
| 3. Magnesium stearate | 1.5 mg |
| 4. Aerosil | 3.0 mg |
| 5. Crospovidone | 4.0 mg |

Ingredients 1, 2 and 5 were pre-mixed for 10 min in a free-fall mixer (Turbula® T10B) and control-screened through a 1.25 mm screen. This mixture was compacted with 70% of ingredients 3 and 4 using a roll compactor and screened with a mesh width of 1.25 mm. The compacted material was mixed with the remaining substances and pressed into tablets.

### Example 6a

### Production of IR Tablets by Means of Direct Compression

In order to produce tablets, the following formulation was used.

| | |
|---|---|
| 1. Intermediate according to Example 3a | 130 mg |
| 2. Calcium hydrogen phosphate | 120 mg |
| 3. Magnesium stearate | 2 mg |
| 4. Aerosil ® | 3 mg |
| 5. Crospovidone | 20 mg |
| 6. Na bicarbonate | 25 mg |

The intermediate from Example 3a was mixed for 15 minutes with calcium hydrogen phosphate, Na bicarbonate and crospovidone in a free-fall mixer (Turbula® T10B) and screened (1.25 mm), after which the remaining two excipients were added and mixed for 5 minutes. The finished mixture was compressed on an EK0-type eccentric press (Korsch).

### Example 6b

### Production of MR Tablets by Means of Direct Compression

In order to produce tablets, the following formulation was used.

| | |
|---|---|
| 1. Intermediate according to Example 3b | 130 mg |
| 2. Calcium hydrogen phosphate | 120 mg |
| 3. Magnesium stearate | 2 mg |
| 4. Aerosil ® | 3 mg |
| 5. Crospovidone | 10 mg |

The intermediate from Example 3b was mixed for 15 minutes with calcium hydrogen phosphate and crospovidone in a free-fall mixer (Turbula® T10B) and screened (1.25 mm), after which the remaining two excipients were added and mixed for 5 minutes. The finished mixture was compressed on an EK0-type eccentric press (Korsch).

1. An intermediate comprising tapentadol in solid, non-crystalline form and a surface stabiliser.

2. The intermediate as claimed in claim 1, comprising tapentadol in the form of a solid solution and a surface stabiliser.

3. The intermediate as claimed in claim 1, comprising tapentadol in amorphous form and a surface stabiliser.

4. The intermediate as claimed in claim 1, characterised in that (1R-2R)-tapentadol hydrochloride or (1R-2R)-tapentadol in the form of the free base is contained.

5. The intermediate as claimed in claim 1, characterised in that the surface stabiliser is a magnesium aluminium silicate, a sugar alcohol, polyvinyl pyrrolidone or a copolymer of vinyl pyrrolidone and vinyl acetate.

6. The intermediate as claimed in claim 1, characterised in that the weight ratio of tapentadol to surface stabiliser is 10:1 to 1:10.

7. The intermediate as claimed in claim 1, comprising tapentadol hydrochloride and magnesium aluminium silicate, wherein the weight ratio of tapentadol hydrochloride to magnesium aluminium silicate is 5:1 to 1:3.

8. The intermediate as claimed in claim 1, characterised in that in addition, it comprises a crystallisation inhibitor based on an inorganic salt, an organic acid, a polymer with a weight-average molecular weight of more than 500,000 g/mol, a silicate or mixtures thereof.

9. A method of preparing an intermediate as claimed in claim 1, comprising the steps of
(a6) dissolving the tapentadol, preferably the crystalline tapentadol, in a solvent or mixture of solvents,
(b6) adding the surface stabiliser and
(c6) removing the solvent or mixture of solvents, wherein tapentadol is adsorbed to the surface of the surface stabiliser in non-crystalline form.

10. The method of preparing an intermediate as claimed in claim 1, comprising the steps of
(a2) dissolving tapentadol and a surface stabiliser in a solvent or mixture of solvents, and
(b2) spray-drying the solution from step (a2).

11. The method of preparing an intermediate as claimed in claim 1, comprising the steps of
(a4) dissolving tapentadol and the surface stabiliser in a solvent or mixture of solvents, and
(b4) freeze-drying the solution from step (a4).

12. An intermediate obtained by the method as claimed in claim 9.

13. A pharmaceutical formulation containing non-crystalline tapentadol in the form of an intermediate as claimed in claim 1, and optionally at least one further pharmaceutical excipient.

14. The pharmaceutical formulation as claimed in claim 13, comprising
non-crystalline tapentadol in the form of an intermediate, wherein the intermediate is screened through a screen with a mesh width of more than 0.71 mm and it is a delayed-release formulation;
or
non-crystalline tapentadol in the form of an intermediate, wherein the intermediate is screened through a screen with a mesh width of 0.71 mm or less and it is an immediate-release formulation;

15. A tablet comprising 50 to 500 mg tapentadol, the tablet having a hardness of 50 to 250 N, a friability of less than 3% and a content uniformity of 95 to 105%, and wherein the administration regarding the active agent tapentadol leads to a Tₘₐₓ of 0.5 to 6 hours, preferably 1 to 5 hours, a Cₘₐₓ of 5 to 200 ng/ml, and an AUC of 40 to 1,000 ng×h/ml.

16. An intermediate obtained by the method as claimed in claim 10.

17. An intermediate obtained by the method as claimed in claim 11.

18. A melt-processing method, comprising the steps of
(a3) mixing tapentadol and a surface stabilizer to form a mixture; and
(b3) melt-processing the mixture under conditions such that there is a transition from crystalline to non-crystalline tapentadol.

19. The melt-processing method as claimed in claim 18, wherein the melt-processing method comprising a melt-extrusion process.
